(19) United States
(12) Patent Application Publication
(10) Pub. No.: US 2012/0053571 A1
(43) Pub. Date: Mar. 1, 2012

(54) FLUID DELIVERY DEVICE WITH ACTIVE AND PASSIVE FLUID DELIVERY

(75) Inventor: Ronald J. Petri, Lake Elmo, MN (US)

(73) Assignee: Medtronic, Inc., Minneapolis, MN (US)

(21) Appl. No.: 13/220,359

(22) Filed: Aug. 29, 2011

Related U.S. Application Data
(60) Provisional application No. 61/378,670, filed on Aug. 31, 2010.

(51) Int. Cl. A61M 37/00

(52) U.S. Cl. 604/891.1

(57) ABSTRACT

The disclosure generally describes fluid delivery devices that include both active and passive fluid delivery. In one example, a therapeutic fluid delivery device includes a first reservoir configured to house a first therapeutic fluid and a second reservoir configured to house a second therapeutic fluid. The first reservoir is configured to passively transfer the first therapeutic fluid to a patient. In addition, the therapeutic fluid delivery device includes a fluid delivery pump configured to actively transfer the second therapeutic fluid from the second reservoir to the patient.

This application claims the benefit of U.S. Provisional Application No. 61/378,670, entitled “FLUID DELIVERY DEVICE WITH ACTIVE AND PASSIVE FLUID DELIVERY,” and filed on Aug. 31, 2010, the entire contents of which are incorporated herein by reference.

### TECHNICAL FIELD

This disclosure generally relates to implantable medical devices and, more particularly, to implantable fluid delivery devices.

### BACKGROUND

A variety of medical devices are used for chronic, i.e., long-term, delivery of fluid therapy to patients suffering from a variety of conditions, such as chronic pain, tremor, Parkinson's disease, epilepsy, urinary or fecal incontinence, sexual dysfunction, obesity, spasticity, or gastroparesis. For example, pumps or other fluid delivery devices can be used for chronic delivery of therapeutic fluids, such as drugs to patients. These devices are intended to provide a patient with a therapeutic output to alleviate or assist with a variety of conditions. Typically, such devices are implanted in a patient and provide a therapeutic output under specified conditions on a recurring basis.

One type of implantable fluid delivery device is a drug infusion device that can deliver a drug or other therapeutic fluid to a patient at a selected site. A drug infusion device may be partially or completely implanted at a location in the body of a patient and deliver a fluid medication through a catheter to a selected delivery site in the body. Drug infusion devices, such as implantable drug pumps, commonly include a reservoir for holding a supply of the therapeutic fluid, such as a drug, for delivery to a site in the patient. The fluid reservoir can be self-sealing and accessible through a port. A pump may be fluidly coupled to the reservoir for delivering the therapeutic fluid to the patient. A catheter provides a pathway for delivering the therapeutic fluid from the pump to a delivery site in the patient.

### SUMMARY

In general, the disclosure describes fluid delivery devices that include both active and passive fluid delivery. In some examples, passive fluid delivery is achieved through a reservoir of pressurized therapeutic fluid. A pressure differential between the reservoir and the patient acts as a driving force to passively deliver fluid from the device to the patient. In some examples, active fluid delivery is achieved through a fluid delivery pump that imparts mechanical energy to a fluid to drive the fluid from device to patient. According to this disclosure, some fluid delivery devices may include multiple fluid reservoirs, e.g., to house different types of fluids or different or similar quantities of the same type of fluid. The fluid delivery device may actively deliver fluid from one reservoir and passively deliver fluid from another reservoir.

In one example, an implantable therapeutic fluid delivery device includes a first reservoir configured to house a first therapeutic fluid and a second reservoir configured to house a second therapeutic fluid. The first reservoir is configured to passively transfer the first therapeutic fluid to a patient. In addition, the therapeutic fluid delivery device includes a fluid delivery pump configured to actively transfer the second therapeutic fluid from the second reservoir to the patient.

In another example, an implantable therapeutic fluid delivery device includes means for housing a first therapeutic fluid, means for housing a second therapeutic fluid, means for passively delivering the first therapeutic fluid to a patient, and means for actively delivering the second therapeutic fluid to the patient.

In an additional example, a method comprises passively delivering a first therapeutic fluid to a patient from a first reservoir configured to house the first therapeutic fluid, and actively delivering a second therapeutic fluid to the patient from a second reservoir configured to house the second therapeutic fluid, wherein an implantable medical device includes the first reservoir and the second reservoir.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example of a fluid delivery system including an implantable fluid delivery device configured to deliver a therapeutic fluid to a patient via a catheter.

FIG. 2 is functional block diagram illustrating an example of the implantable fluid delivery device of FIG. 1.

FIG. 3 is a functional block diagram illustrating an example of an external programmer shown in FIG. 1.

FIG. 4A is a top view of an example implantable fluid delivery device.

FIG. 4B is a cross-sectional side view of the example implantable fluid delivery device of FIG. 4A.

FIG. 4C is a side view illustrating an example multilayer structure of an example reservoir of the example fluid delivery device of FIG. 4B.

FIG. 5 is a flow chart illustrating an example method of delivering therapeutic fluid with an example implantable fluid delivery device.

FIG. 6 is a plot illustrating example fluid delivery rates provided by the implantable fluid delivery device of FIG. 1 versus time.

### DETAILED DESCRIPTION

Fluid delivery devices can be configured to treat a variety of different medical conditions. In one example, a fluid delivery device may be implanted in the body of a patient to deliver a fluid, such as a drug or other therapeutic agent, through a catheter to one or more delivery sites within the body of the patient. The implantable fluid delivery device may include a reservoir for storing the therapeutic fluid prior to delivery to a patient. The implantable fluid delivery device may also include a fluid delivery pump. During operation, the fluid delivery pump may draw therapeutic fluid from the reservoir, pressurize therapeutic fluid in the delivery pump, and then discharge the pressurized fluid for delivery to the patient. In some examples, the fluid delivery pump is operable to deliver therapeutic fluid at a variety of different, selectable fluid delivery rates. In other examples, the fluid delivery pump is configured to deliver therapeutic fluid at a constant dosing rate.

In some examples, the type of fluid therapy required by a patient will be dictated by the patient's specific medical condition. With some medical conditions, a fluid delivery device that delivers a single therapeutic fluid at a fixed dosing rate may be sufficient to treat a patient's medical condition. On the other hand, other medical conditions may require more complex fluid therapies to achieve an efficacious therapeutic result. For example, some medical conditions may require a fluid delivery device that delivers fluid at a variety of different rates, e.g., to respond to changing symptoms, times, or activities of a patient. As another example, some medical conditions may require delivery of multiple therapeutic fluids, e.g., to address different medical conditions or to effectively treat a single condition. As a further example, some medical conditions may require delivery of a therapeutic fluid to different regions of the body of a patient.

To accommodate more complex fluid therapies, a physician may consider a variety of different fluid delivery strategies. With some patients, multiple fluid delivery devices, e.g., that house different therapeutic fluids or that operate at different fluid delivery rates, may be implanted into a body of the patient. However, multiple implantable fluid delivery devices can be expensive and can require the patient to undergo multiple surgical procedures. In addition, multiple implantable fluid delivery devices may require multiple implant pockets and/or tunneling paths, consuming more space within the patient's body. In other examples, a patient may receive a single, complex fluid delivery device that is capable of delivering a complex fluid therapy regime. Once implanted in the body of the patient, however, the complex fluid delivery device may consume more energy than a comparatively simpler fluid delivery device, which may reduce the service life of the fluid delivery device, particularly in the case of a device with a non-rechargeable power source.

In accordance with the techniques described in this disclosure, a fluid delivery device with both active and passive fluid delivery is provided. The fluid delivery device includes a first reservoir configured to house a pressurized reservoir of therapeutic fluid. The first reservoir may be configured to passively transfer pressurized therapeutic fluid to a patient. A pressure differential between the first reservoir and the body of the patient acts as a driving force to transfer therapeutic fluid from the reservoir to the patient, e.g., at a substantially constant rate. Because the therapeutic fluid does not pass through a fluid delivery pump, the therapeutic fluid is considered to be passively delivered. In addition to passive fluid delivery, however, a fluid delivery device according to this disclosure also may include a fluid delivery pump to actively deliver therapeutic fluid. In some examples, the fluid delivery pump draws second therapeutic fluid from a second reservoir different from the first, pressurized reservoir housing the first therapeutic fluid.

Hence, the first reservoir may be configured to house or otherwise contain a first therapeutic fluid and the second reservoir may be configured to house or otherwise contain a second therapeutic fluid. The first and second therapeutic fluids may be the same type of fluid or different types of fluid. The first reservoir may be configured to passively transfer the first therapeutic fluid to a patient. For example, the first reservoir may be pressurized to passively deliver the first therapeutic fluid. The first therapeutic fluid may be pressurized for passive delivery, e.g., at a substantially constant rate. The second reservoir may be coupled to a fluid delivery pump that is configured to actively transfer the second therapeutic fluid from the second reservoir to the patient. Hence, a pressure of a pressurized first therapeutic fluid may provide force to transfer the pressurized first therapeutic fluid from the first reservoir to the patient, and a fluid delivery pump may provide force to transfer the second therapeutic fluid from the second reservoir to the patient. In an example method for delivering therapeutic fluid, passively delivering the first therapeutic fluid may include providing a pressure on the first therapeutic fluid to provide force to transfer the first therapeutic fluid to the patient, and actively delivering the second therapeutic fluid may include applying a fluid delivery pump to provide force to transfer the second therapeutic fluid to the patient. An implantable medical device may contain the first reservoir, the second reservoir, and the fluid delivery pump.

In some examples, the first and second therapeutic fluids may be delivered via one or more catheters or other fluid delivery elements. The first and second therapeutic fluids in the first reservoir and the second reservoir, respectively, may be the same or may be different. In some examples, active and passive fluid delivery channels deliver therapeutic fluid to the same or different target therapy sites within the body of the patient. In any event, because the device includes both active and passive therapeutic fluid delivery, a reliable, energy efficient, multifunctional device is provided.

In some examples according to this disclosure, the fluid delivery device may include a controllable valve interposed between the pressurized reservoir configured to house the therapeutic fluid and the patient. The valve may be selectively actuated to restrict or close a fluid pathway between the reservoir and the patient. As a result, the fluid delivery device may provide flow control from the passive delivery channel.

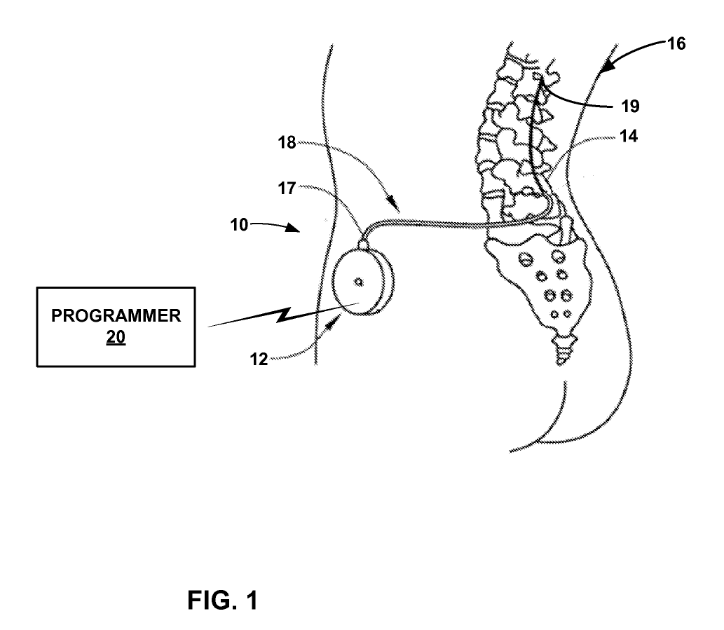

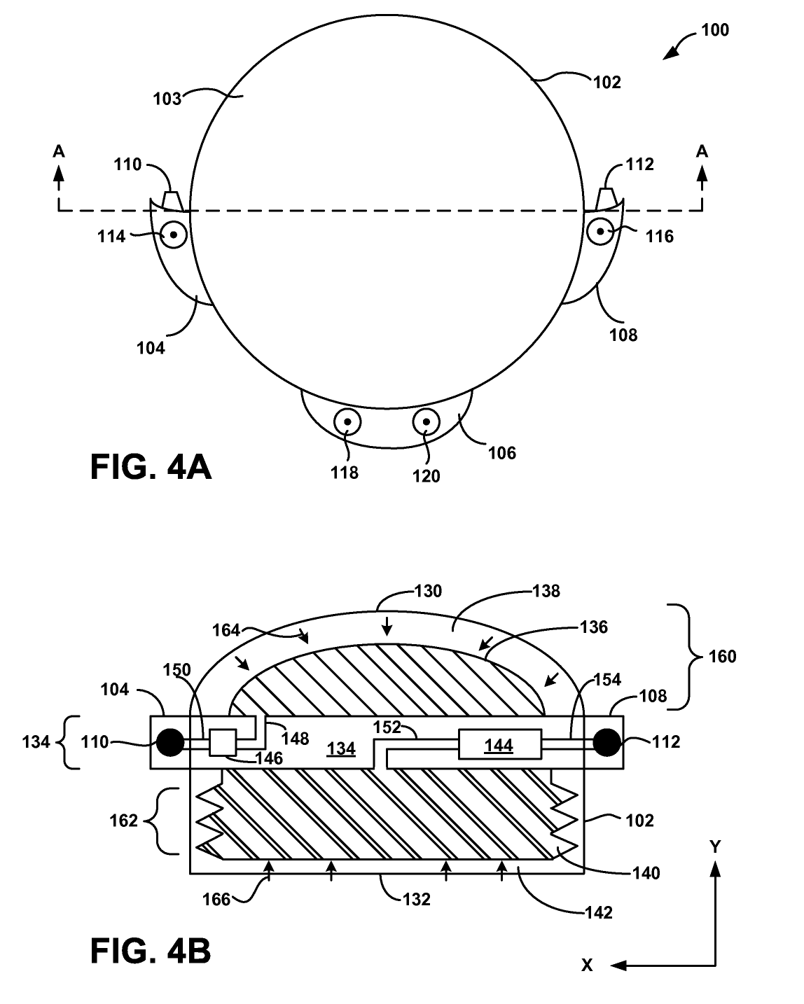

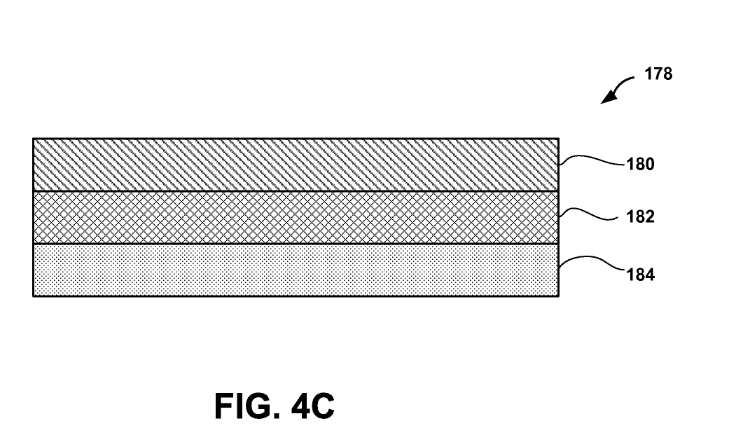

Conceptual details for an example fluid delivery device will be described in greater detail with reference to FIGS. 4A-C. However, an example fluid delivery system including an implantable fluid delivery device and external programmer will first be described with reference to FIGS. 1-3.

FIG. 1 is a conceptual diagram illustrating an example of a therapy system 10, which includes implantable medical device (IMD) 12, catheter 18, and external programmer 20. IMD 12 is connected to at least one catheter 18 to deliver at least one therapeutic fluid, e.g. a pharmaceutical agent, pain relieving agent, anti-inflammatory agent, gene therapy agent, or the like, to a target site within patient 16. In some examples, IMD 12 includes a single outer housing that may be constructed of a biocompatible material that resists corrosion and degradation from bodily fluids. The biocompatible material may include titanium or biologically inert polymers.

In other examples, IMD 12 may include a first housing that contains a first reservoir of therapeutic fluid and a second housing that contains a second reservoir of therapeutic fluid. In some of these examples, IMD 12 may include a member that at least partially encapsulates the first housing and the second housing. The first housing and the second housing may be constructed of a biocompatible material that resists corrosion and degradation from bodily fluids, such as titanium or a biologically inert polymer. The member may be, for example, formed of a biologically inert polymer, which may be flexible. For example, the member may be formed of silicone or polyurethane. The member may couple the first housing and the second housing to form a single IMD 12.

IMD 12 may be implanted within a subcutaneous pocket relatively close to the therapy delivery site. For example, as shown in FIG. 1, IMD 12 may be implanted within an abdomen of patient 16. In other examples, IMD 12 may be implanted within other suitable sites within patient 16, which may depend, for example, on the target site within patient 16 for the delivery of the therapeutic fluid. In still other examples, device 12 may be external to patient 16 with a percutaneous catheter connected between device 12 and the target delivery site within patient 16. In these examples, device 12 is not an implantable medical device but rather an external medical device.

As described in greater detail below, IMD 12 is configured for active fluid delivery and passive fluid delivery. In some examples, passive fluid delivery is achieved through a pressurized reservoir of therapeutic fluid. A pressure differential between the reservoir and patient 16 acts as a driving force to deliver therapeutic fluid from IMD 12 to patient 16. In some examples, active fluid delivery is achieved through a fluid delivery pump that imparts mechanical energy to a therapeutic fluid to drive the therapeutic fluid from IMD 12 to patient 16. In various examples, a therapeutic fluid may be actively and passively delivered through the same catheter 18 or, alternatively, through separate fluid pathways, e.g., in separate catheters or separate lumens of the same catheter.

IMD 12 delivers a therapeutic fluid from a reservoir (not shown in FIG. 1) to patient 16 through catheter 18 from proximal end 17 coupled to IMD 12 to distal end 19 located proximate to the target site. Example therapeutic fluids that may be delivered by IMD 12 include, e.g., insulin, morphine, hydromorphone, bupivacaine, clonidine, other analgesics, baclofen and other muscle relaxers and antispastic agents, genetic agents, proteins, antibiotics, nutritional fluids, hormones or hormonal drugs, gene therapy drugs or agents, anticoagulants, cardiovascular medications or chemotherapeutics.

Catheter 18 can comprise a unitary catheter or a plurality of catheter segments connected together to form an overall catheter length. In addition, catheter 18 may be a single-lumen catheter or a multi-lumen catheter. Catheter 18 may be coupled to IMD 12 either directly or with the aid of a catheter extension (not shown in FIG. 1). In the example shown in FIG. 1, catheter 18 extends from the implant site of IMD 12 to one or more targets proximate to spinal cord 14, e.g., within an intrathecal space or epidural space. Catheter 18 is positioned such that one or more fluid delivery outlets (not shown in FIG. 1) of catheter 18 are proximate to the targets within patient 16. In the example of FIG. 1, IMD 12 delivers a therapeutic fluid through catheter 18 to one or more targets proximate to spinal cord 14.

IMD 12 can be configured for intrathecal drug delivery into the intrathecal space, as well as epidural delivery into the epidural space, both of which surround spinal cord 14. In some examples, multiple catheters may be coupled to IMD 12 to target the same or different nerve sites or other tissue sites within patient 16, or catheter 18 may include multiple lumens to deliver multiple therapeutic fluids to the patient. Therefore, although the target site shown in FIG. 1 is proximate to spinal cord 14 of patient 16, other applications of therapy system 10 may include alternative target delivery sites in addition to or in lieu of the spinal cord 14 of the patient 16. For example, therapy system 10 may be configured to deliver single or multisite deep-brain infusion therapy. As another example, therapy system 10 may be configured to deliver therapeutic fluid to the bloodstream.

Programmer 20 is an external computing device that is configured to communicate with IMD 12 by wireless telemetry as needed, such as to provide or retrieve therapy information or control aspects of therapy delivery (e.g., modify the therapy parameters such as rate or timing of delivery, turn IMD 12 on or off, and so forth) from IMD 12 to patient 16. In some examples, programmer 20 may be a clinician programmer that the clinician uses to communicate with IMD 12 and to program therapy delivered by IMD 12. Alternatively, programmer 20 may be a patient programmer that allows patient 16 to view and modify therapy parameters associated with therapy programs. The clinician programmer may include additional or alternative programming features compared to the patient programmer. For example, more complex or sensitive tasks may only be allowed by the clinician programmer to prevent patient 16 from making undesired or unsafe changes to the operation of IMD 12. Programmer 20 may be a handheld or other dedicated computing device, or a larger workstation or a separate application within another multi-function device.

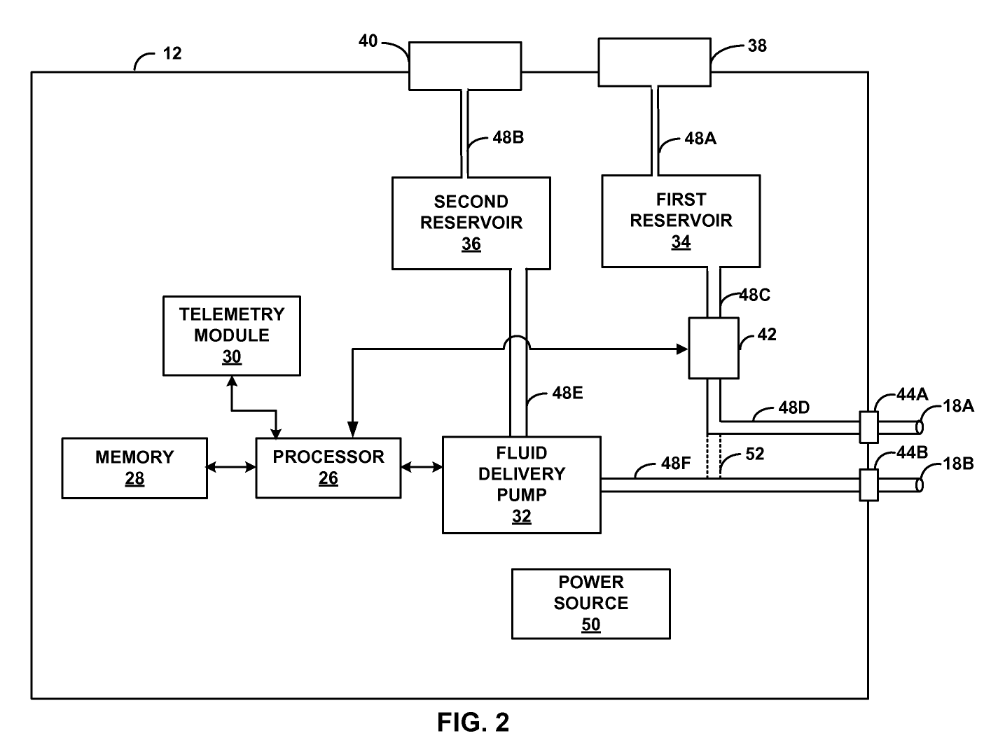

FIG. 2 is a functional block diagram illustrating components of an example of IMD 12, which includes processor 26, memory 28, telemetry module 30, fluid delivery pump 32, first reservoir 34, second reservoir 36, first reservoir inlet port 38, second reservoir inlet port 40, first reservoir discharge valve 42, first catheter port 44A, second catheter port 44B, first catheter 18A, second catheter 18B, internal fluid pathways 48A-48F (collectively, “internal fluid pathways 48”), and power source 50. Processor 26 is communicatively connected to memory 28, telemetry module 30, fluid delivery pump 32, and first reservoir discharge valve 42. Fluid delivery pump 32 is connected to second reservoir 36 through fluid pathway 48. First reservoir 34 and second reservoir 36 are connected to first reservoir inlet port 38 and second reservoir inlet port 40 through fluid pathways 48A and 48B, respectively. First reservoir 34 discharges through fluid pathway 48C, first reservoir discharge valve 42, fluid pathway 48D and first catheter port 44A, which is connected to first catheter 18A. Second reservoir 36 discharges through fluid pathway 48E, fluid delivery pump 32, fluid pathway 48F and second catheter port 44B, which is connected to second catheter 18B.

IMD 12 also includes power source 50, which is configured to deliver operating power to various components of the IMD. In some examples, IMD 12 may include a single reservoir in fluid communication with both first catheter port 44A and second catheter port 44B, instead of first reservoir 34 and second reservoir 36. In other examples, IMD 12 may include more than two reservoirs 34, 36 (e.g., three, four, five or more reservoirs) for storing more than two types of therapeutic fluid or for storing different amounts of therapeutic fluid, or for storing the same type of therapeutic fluid in multiple reservoirs, e.g., in the same or different quantities. In additional examples, IMD 12 may include a different number of catheter ports 44A, 44B configured to connect to a different number of catheters 18A, 18B. According to one example, first reservoir 34 in IMD 12 discharges through fluid pathway 48C, first reservoir discharge valve 42, fluid pathway 52 and catheter port 44B, instead of discharging through catheter port 44A. However, for ease of description, IMD 12 in FIG. 2 includes two reservoirs 34, 36 in fluid communication with two separate catheter ports 44A, 44B.

During operation of IMD 12, fluid is delivered from first reservoir 34 and second reservoir 36, e.g., either simultaneously or at separate times. To passively deliver a dose of fluid from first reservoir 34, processor 26 controls first reservoir discharge valve 42, e.g., with the aid of instructions stored in memory 28 or upon receiving a command via telemetry module 30. Processor 26 opens first reservoir discharge valve 42, allowing fluid to migrate from first reservoir 34 to patient 16 via catheter 18A under influence of the pressure differential between reservoir 34 and catheter 18A. Fluid may flow from first reservoir 34 at a substantially constant rate, e.g., based on a substantially constant fluid pressure in the reservoir, while discharge valve 42 is open. In some examples, actuating first reservoir discharge valve 42 to different positions may control the rate, and hence the dose, of therapeutic fluid delivered to patient 16 from first reservoir 12. For example, actuating first reservoir discharge valve 42 to different positions may change the size of a discharge orifice, thereby controlling the flow rate through first reservoir discharge valve 42. While IMD 12 in the example of FIG. 2 includes first reservoir discharge valve 42 to provide dosing control, in other examples, IMD 12 does not include first reservoir discharge valve 42. In these examples, IMD 12 delivers fluid from first reservoir 34 to patient 16 immediately upon filling first reservoir 34. Fluid may be continuously delivered from first reservoir 34 until, e.g., first reservoir 34 is empty. Independent of whether IMD 12 includes first reservoir discharge valve 42, in some examples, IMD 12 has an outlet orifice, e.g., defined by fluid pathway 48C or 48D, first reservoir discharge valve 42, or catheter port 44A, that is sized to provide a fluid restriction to meter the flow of fluid passively delivered from first reservoir 34. In additional examples, IMD 12 may include a separate restrictor, e.g., to restrict flow out of first reservoir 34, in addition to or in lieu of a restriction provided by fluid pathway 48C or 48D, first reservoir discharge valve 42, or catheter port 44A.

To actively deliver a dose of fluid from second reservoir 36, processor 26 controls fluid delivery pump 32. Instructions stored in memory 28 specify parameters for controlling fluid delivery pump 32, e.g., for cycling fluid delivery pump 32 on and off, or for controlling the rate at which fluid delivery pump 32 delivers fluid. In this manner, IMD 12 provides active control of fluid delivery from second reservoir 36. In some examples, IMD 12 includes one or more valves interposed between second reservoir 36 and fluid delivery pump 32, or between fluid delivery pump 32 and catheter port 44B. Accordingly, processor 26 may also actuate one or more valves to facilitate control of fluid delivery from second reservoir 36.

In various examples, instructions executed by processor 26 may define therapy programs that specify delivery of different fluids housed in first reservoir 34 and second reservoir 36, e.g., at different times or different rates. The programs may alternatively specify a schedule of different delivery parameters by which IMD 12 delivers therapy to patient 16. In some examples, various instructions, such as instructions that define therapy programs, may be stored in a memory of an external device communicatively connected to IMD 12. In one example, therapy program instructions are stored in a memory of programmer 20 and communicated to processor 26 via telemetry module 30.

In some examples, therapeutic dosing is specified in mass of drug or dosing agent delivered per unit of time (micrograms per unit of time). Fluid delivery systems typically control fluid flow and hence the volume of fluid per unit time (microliter per unit of time). The dose in mass of therapeutic agent per unit of time intended by the clinician to be delivered to the patient is converted to flow rate (microliter per unit of time) based upon the concentration of the dosing agent in the fluid being delivered (micrograms of dosing agent per microliter of fluid being delivered to the patient). This conversion may be carried out in the programmer, by the processor in the IMD or by a combination of the components in the fluid delivery system.

In some examples, instructions may specify a dosing rate of therapeutic fluid (e.g., in microliters per unit of time) to be actively delivered from second reservoir 36. Processor 26 may control the infusion rate of fluid delivery pump 32 (e.g., in a volume of fluid per unit of time) according to the instructions to actively deliver therapeutic fluid from second reservoir 36 at the specified dosing rate. In another example, instructions may specify a dosing rate of therapeutic fluid to be passively delivered from first reservoir 34. Processor 26 may control the actuation of discharge valve 42 according to the instructions to passively deliver therapeutic fluid from first reservoir 34 at the specified dosing rate.

In some examples, a therapy program stored on memory 28 and executed by processor 26 defines one or more therapeutic fluid doses to be delivered from first reservoir 34 and/or second reservoir 36 to patient 16 through catheters 18A, 18B by IMD 12. A dose of therapeutic fluid generally refers to a total amount of therapeutic fluid, e.g., in volumetric units, delivered over a total amount of time, e.g., twenty-four hour period.

In some examples, a sufficient amount of the fluid should be administered in order to have a desired therapeutic effect, such as pain relief. However, the amount of the therapeutic fluid delivered to the patient may be limited to a maximum amount, such as a maximum daily amount, in order to avoid potential side effects. Therapy program parameters specified by a user, e.g., via programmer 20, may include the type of therapeutic fluid (e.g., when different types of fluid are housed in reservoir 34 and 36), fluid volume per dose, dose time period, maximum dose for a given time interval e.g., daily, or the like. While IMD 12 may accommodate therapy parameters to control fluid delivery from both first reservoir 34 and second reservoir 36, in some examples, IMD 12 may not accommodate the same number or type of therapy parameters for fluid delivery from first reservoir 34 as for fluid delivery from second reservoir 36. In this regard, IMD 12 may provide less control for passive fluid delivery from first reservoir 34 than for active fluid delivery from second reservoir 36.

The manner in which a dose of therapeutic fluid is delivered to patient 16 by IMD 12 may also be defined in the therapy program. For example, processor 26 of IMD 12 may be programmed to deliver a dose of therapeutic fluid according to a schedule that defines different rates at which the fluid is to be delivered at different times during the dose period, e.g. a twenty-four hour period. The therapeutic fluid rate refers to the amount, e.g., in volume, of therapeutic fluid delivered over a unit period of time, which may change over the course of the day as IMD 12 delivers the dose of fluid to patient 16. As another example, processor 26 of IMD 12 may be programmed to deliver a dose of different therapeutic fluids, e.g., according to a schedule that defines times and rates for delivering different therapeutic fluids. In one example, processor 26 of IMD 12 is configured to mix different therapeutic fluids in a mixing chamber (not shown in FIG. 2) in fluid communication with first reservoir 34 and second reservoir 36, e.g., based on mixing ratios specified in a look-up table or per instructions stored in memory 28, to deliver a composite therapeutic fluid based on therapeutic fluids housed in both reservoir 34 and reservoir 36. In another example, fluid pathway 48D and/or fluid pathway 48F includes a one-way valve, and processor 26 of IMD 12 is configured to mix a higher pressure therapeutic fluid with a lower pressure therapeutic fluid in the fluid pathway corresponding to the lower pressure therapeutic fluid. In various examples, IMD 12 may be configured to deliver therapeutic fluid solely from first reservoir 34, solely from second reservoir 36, to switch between delivering therapeutic fluid from first reservoir 34 and second reservoir 36, or to simultaneously deliver therapeutic fluid from first reservoir 34 and second reservoir 36.

As one example, IMD 12 could be programmed to continuously deliver therapeutic fluid from first reservoir 34 while intermittently delivering fluid from second reservoir 36. A continuous dose of fluid delivered at a substantially constant rate may be referred to as a basal dose or basal rate. According to one example, IMD 12 could be programmed to passively deliver a basal dose of approximately 10 microliters per hour from first reservoir 34. Processor 26 can actuate first reservoir discharge valve 42 to a position that corresponds to a basal fluid delivery rate of approximately 10 microliters per hour, e.g., 50 percent open. Upon opening, a pressure differential between first reservoir 34 and patient 16 forces fluid from first reservoir 34 through catheter 18A into patient 16. In the event the therapy program prescribes this fluid delivery rate for a twenty four hour period and assuming IMD 12 delivers no patient activated boluses or other boluses during the period of time, the dose of fluid delivered to patient 16 by IMD 12 will be 240 microliters (per twenty four hours).

In addition to passively delivering a basal dose from first reservoir 34, however, IMD 12 can also be programmed to actively deliver fluid from second reservoir 36 at various times, e.g., either to provide a supplemental amount of the same therapeutic fluid housed in first reservoir 34 or to provide a different therapeutic fluid. In response to instructions stored on memory 28 or a command received via telemetry module 30, processor 26 controls fluid delivery pump 32 to draw fluid from second reservoir 36 and deliver fluid to patient 16 via catheter 18B. In one example, IMD 12 can be programmed to deliver fluid from second reservoir 36 at a rate of 15 microliters per hour between 7:00 AM and 10:00 AM, and 5 microliters per hour between 4:00 PM and 10:00 PM. When combined with the basal dose of 10 microliters per hour described above, the dose of fluid delivered to patient 16 by IMD 12 will be 315 microliters (per twenty four hours). In different examples, the therapy program may include other parameters, including, e.g., definitions of priming and patient boluses, as well as minimum time intervals between successive patient activated boluses, sometimes referred to as lock-out intervals.

Therapy programs may be a part of a program group, where the group includes a number of therapy programs. Memory 28 of IMD 12 or a memory associated with programmer 20 may store one or more therapy programs, as well as instructions defining the extent to which patient 16 may adjust therapy parameters, switch between therapy programs, or undertake other therapy adjustments. Patient 16 or a clinician may select and/or generate additional therapy programs for use by IMD 12, e.g., via programmer 20 at any time during therapy or as designated by the clinician.

Components described as processors within IMD 12, external programmer 20, or any other device described in this disclosure may each include one or more processors, such as one or more microprocessors, digital signal processors (DSPs), application specific integrated circuits (ASICs), field programmable gate arrays (FPGAs), programmable logic circuitry, or the like, either alone or in any suitable combination. Other components may be formed by suitable electrical and/or mechanical hardware elements, in combination with software or firmware, as appropriate.

In one example, processor 26 of IMD 12 is programmed to deliver a dose of therapeutic fluid to patient 16, which is defined in memory 28 of the device by a volume of therapeutic fluid delivered to the patient in one day. IMD 12 is also programmed according to a therapy schedule such that different fluids housed in first reservoir 34 and second reservoir 36 are delivered at different rates at different times during the day, which may be stored in the device memory, e.g., as a look-up table associating different fluids, different fluid rates and different times of the day.

Upon instruction from processor 26, first reservoir discharge valve 42 actuates open to allow fluid from first reservoir 34 to transfer though fluid pathways 48C and 48D to catheter 18A to patient 16. Further, upon instruction from processor 26, fluid delivery pump 32 activates to draw fluid from second reservoir 36 through fluid pathway 48E and to deliver the fluid through fluid pathway 48F to catheter 18B to patient 16, e.g., in accordance with the program stored on memory 28.

Fluid pathways 48 in IMD 12 may be segments of tubing or ducts within IMD 12 that allow fluid to be conveyed through IMD 12. In some examples, fluid pathways 48 may be machined or cast into IMD 12. Fluid pathways 48 may be created from a biocompatible material, e.g., titanium, stainless steel, or biologically inert polymer, and sized, e.g., to accommodate desired flow rates in IMD 12.

First catheter port 44A and second catheter port 44B are apertures defined in the housing (or housings) of IMD 12. First catheter port 44A and second catheter port 44B are configured to allow fluid communication between first reservoir 34 and second reservoir 36, respectively, and patient 16. In some examples, first catheter port 44A is configured to connect to first catheter 18A, while second catheter port 44B is configured to connect second catheter 18B. In various examples, IMD 12 is configured to deliver fluid from first reservoir 34 and second reservoir 36 through separate fluid lumens, e.g., different catheters 18A, 18B or different lumens of a multi-lumen catheter. In this manner, IMD 12 can be configured to provide isolated fluid pathways from first reservoir 34 and second reservoir 36 to patient 16, which may be desirable for various reasons including, e.g., to prevent mixing of incompatible fluids or to allow simultaneous fluid delivery to two separate regions of patient 16.

First reservoir discharge valve 42 is configured to control fluid communication between first reservoir 34 and patient 16. First reservoir discharge valve 42 may be any device that regulates the flow of a fluid by opening, closing, or partially obstructing a fluid pathway. In some examples, first reservoir discharge valve 42 may actuate to any position between fully closed and fully open, e.g., providing a continuous range of valve settings between 0 percent open and 100 percent open. In other examples, first reservoir discharge valve 42 may actuate to a discrete number of settings. In one example, first reservoir discharge valve 42 actuates to five discrete settings: fully closed, one-quarter open, half-open, three-quarters open, and fully open. In another example, first reservoir discharge valve 42 actuates to two discrete settings: fully closed and fully open. In various examples, first reservoir discharge valve 42 may be a micro-machined valve, such as micro-machined diaphragm valve, ball valve, check valve, gate valve, slide valve, piston valve, rotary valve, shuttle valve, or the like. First reservoir discharge valve 42 may include an actuator, such as a pneumatic actuator, electrical actuator, hydraulic actuator, or the like. In another example, first reservoir discharge valve 42 includes a solenoid, piezoelectric element, or similar feature to convent electrical energy into mechanical energy to mechanically open and close valve 42. First reservoir discharge valve 42 may include a limit switch, proximity sensor, or other electromechanical device to provide confirmation that valve 42 is actuated to a specific position.

In yet additional examples according to the disclosure, IMD 12 may not include first reservoir discharge valve 42 but may instead include a restrictor or other metering device that does not actuate between first reservoir 34 and patient 16. For example, first reservoir discharge valve 42 may be replaced by a restrictor between fluid pathways 48C and 48D. The restrictor may be a physical component with a fixed cross-sectional area, which may or may not be less than the cross-sectional area of fluid pathways 48C and/or 48D. The restrictor may control fluid flow from first reservoir 34 by restricting the amount of fluid passing through first catheter port 44A. A restrictor or other metering device that does not actuate may present fewer failure modes than an actively controllable valve and may, in some examples, extend the service life of IMD 12.

First reservoir 34 and second reservoir 36 are generally sized to house enough fluid to allow patient 16 to receive therapeutic dosing without continuously refilling the reservoirs. In some examples, first reservoir 34 and second reservoir 36 are each sized based, e.g., on the shelf-life of the fluid expected to be housed in reservoir 34, 36, or the anticipated delivery rate of the fluid expected to be housed in reservoir 34, 36. In one example, first reservoir 34 and second reservoir 36 each may house between approximately 5 milliliters and approximately 120 milliliters. In some examples, first reservoir 34 and second reservoir 36 are the same size, while in other examples, first reservoir 34 and second reservoir 36 are different sizes.

First reservoir 34 and second reservoir 36 may house the same therapeutic fluid, e.g., in similar or different quantities and/or in similar or different concentrations, to provide therapy dosing flexibility. Alternatively, first reservoir 34 and second reservoir 36 may house different therapeutic fluids, e.g., to achieve different therapeutic effects or to provide different fluid storage conditions, such as acidic and basic pH storage conditions. In various examples, first reservoir 34 and second 36 may be arranged in numerous locations within IMD 12 including, e.g., a stacked arrangement (e.g., one on top of another) or a coplanar arrangement (e.g., side-by-side) to minimize the overall thickness of IMD 12.

As described above, first reservoir 34 is configured to house a therapeutic fluid to passively deliver the therapeutic fluid to patient 16. In some examples, first reservoir 34 is configured to house a therapeutic fluid pressurized between approximately one atmosphere of pressure (i.e., about 14.7 pounds per square inch (psia)) and approximately 2 atmospheres of pressure (i.e., about 29.4 psia), such as, e.g., approximately 1.5 atmospheres of pressure (i.e., about 22 psia). In some examples, first reservoir 34 is configured to house a therapeutic fluid pressurized to at least 1.5 atmospheres of pressure (i.e., about 22 psia). Other pressures are possible, however, and pressures may vary based on a variety of factors such as, e.g., an orifice size provided by first reservoir discharge valve 42 or another restrictor, desired therapeutic fluid deliver rates, and elevations (i.e., altitude above sea level) over which patient 16 is expected to travel.

In some examples, first reservoir 34 is configured to passively transfer therapeutic fluid to patient 16 at a substantially constant rate. As used herein, the phrase “substantially constant rate” means that the rate at which fluid is delivered from first reservoir 34 to patient 16 varies by less than or equal to twenty percent such as, e.g., less than or equal to ten percent from a time when first reservoir 34 is three-quarters full until a time when first reservoir 34 is one-quarter full. In various examples, IMD 12 may be configured to passively deliver therapeutic fluid from first reservoir 34 at a substantially constant rate of between approximately 5 microliters per day and approximately 1500 microliters per day, such as, e.g., between approximately 24 microliters per day and approximately 1000 microliters per day. In some examples, IMD 12 may be configured to passively deliver therapeutic fluid from first reservoir 34 at a rate higher than approximately 1500 microliters per day. For example, higher therapeutic fluid delivery rates may be desirable for some therapies, such as chemotherapy. First reservoir 34 may transfer fluid to patient 16 at a substantially constant rate by maintaining therapeutic fluid in first reservoir 34 at a substantially constant pressure and by maintaining a substantially constant orifice size through first reservoir discharge valve 42. In some examples, IMD 12 includes a biasing means to control the pressure of therapeutic fluid in first reservoir 34. In different examples, biasing means may include, e.g., a spring, piston, pressurized gas, or similar biasing means. An example configuration for first reservoir 34 is described in greater detail with respect to FIGS. 4A-C below.

IMD 12 includes fluid delivery pump 32 for actively delivering fluid to patient 16. Fluid delivery pump 32 can be any mechanism that supplies mechanical force to deliver a therapeutic fluid in some metered or other desired flow dosage to the therapy site within patient 16 from second reservoir 36 via implanted catheter 18B. In various examples, fluid delivery pump 32 may be an axial pump, a centrifugal pump, a pusher plate pump, a piston-driven pump, a peristaltic pump, or other means for moving fluid through fluid pathway 48F and catheter 18B. In one example, fluid delivery pump 32 is an electromechanical pump that delivers fluid by the application of pressure generated by a piston that moves in the presence of a varying magnetic field and that is configured to draw fluid from second reservoir 36 and pump the fluid through fluid pathway 48F and catheter 18B to patient 16. In another example, fluid delivery pump 32 is a squeeze pump that squeezes a fluid pathway in a controlled manner, e.g., such as a peristaltic pump, to progressively move fluid from second reservoir 36 to the distal end of catheter 18B and then into patient 16 according to parameters specified by the therapy program stored on memory 28 and executed by processor 26.

Periodically, fluid may need to be percutaneously added or withdrawn from IMD 12. Fluid may need to be withdrawn from first reservoir 34 and/or second reservoir 36 if a clinician wishes to replace an existing fluid with a different fluid or a similar fluid with different concentrations of therapeutic agents. Fluid may also need to be added to first reservoir 34 and/or second reservoir 36 if all therapeutic fluid has been or will be delivered to patient 16. First inlet port 38 and second inlet port 40 provide access for adding or withdrawing fluid from IMD 12. First inlet port 38 and second inlet port 40 are located on a peripheral surface of a housing (or housings) of IMD 12. First inlet port 38 is in fluid communication with first reservoir 34 via fluid pathway 48A, while second inlet port 40 is in fluid communication with second reservoir 36 via fluid pathway 48B. First inlet port 38 and second inlet port 40 may each include a self-sealing membrane to prevent loss of therapeutic fluid delivered to first reservoir 34 or second reservoir 36. For example, after a percutaneous delivery system, e.g., a hypodermic syringe with fluid delivery needle, penetrates the membrane of either first inlet port 38 or second inlet port 40, the membrane may seal shut when the needle is removed.

In some examples, first reservoir 34 and second reservoir 36 are both accessible through a single inlet port, e.g., a single inlet port that includes a controllable valve to controllably direct fluid to either first reservoir 34 or second reservoir 36, instead of a separate first inlet port 38 and second inlet ports 40. Example inlet ports are described in commonly-assigned U.S. Provisional Patent Application No. 61/376,827 to James M. Haase, entitled “FLUID DELIVERY DEVICE REFILL ACCESS,” and filed on Aug. 25, 2010, and commonly-assigned U.S. Provisional Patent Application No. 61/376,835 to Reginald D. Robinson et al., entitled “DRUG INFUSION DEVICE WITH CONTROLLABLE VALVE,” and filed on Aug. 25, 2010. The entire contents of these applications are incorporated herein by reference.

Awareness of different properties within IMD 12 including, e.g., fluid flow rates, pressures, temperatures, volumes, and the like, may be desirable to monitor the operation of IMD 12. Consequently, IMD 12, in various examples, may include at least one sensor (not shown) to monitor properties within IMD 12. The at least one sensor may be arranged in a number of locations within IMD 12, including, e.g., in first reservoir 34, second reservoir 36, or one or more of fluid pathways 48. In some examples, the at least one sensor is configured to measure a fluid characteristic in IMD 12. In some examples, the at least one sensor may include a pressure sensor, flow sensor, pH sensor, temperature sensor or the like. In other examples, the at least one sensor may be configured to measure a characteristic of the patient in IMD 12 such as, e.g., movement via an accelerometer. In any event, the at least one sensor may generate a signal that is transmitted to processor 26 for, e.g., analysis and storage in memory 28.

Memory 28 may store program instructions and related data that, when executed by processor 26, cause IMD 12 and processor 26 to perform the functions attributed to them in this disclosure. For example, memory 28 of IMD 12 may store instructions for execution by processor 26 including, e.g., therapy programs, programs for actuating first reservoir discharge valve 42, and any other information regarding therapy delivered to patient 16 and/or the operation of IMD 12. Memory 28 may include separate memories for storing instructions, patient information, therapy parameters, therapy adjustment information, dosing schedules, program histories, and other categories of information such as any other data that may benefit from separate physical memory modules. Therapy adjustment information may include information relating to timing, frequency, rates and amounts of patient boluses or other permitted patient modifications to therapy.

At various times during the operation of IMD 12 to treat patient 16, communication to and from IMD 12 may be necessary to, e.g., change therapy programs, adjust parameters within one or more programs, configure or adjust a particular bolus, or to otherwise download information to or from IMD 12. Accordingly, IMD 12 includes telemetry module 30. Processor 26 controls telemetry module 30 to wirelessly communicate between IMD 12 and other devices including, e.g., programmer 20. Telemetry module 30 in IMD 12, as well as telemetry modules in other devices described in this disclosure, such as programmer 20, can be configured to use RF communication techniques to wirelessly send and receive information to and from other devices respectively according to standard or proprietary telemetry protocols. In addition, telemetry module 30 may communicate with programmer 20 via passive or proximal inductive interaction between IMD 12 and the external programmer. Telemetry module 30 may send information to external programmer 20 on a continuous basis, at periodic intervals, or upon request from the programmer.

Power source 50 delivers operating power to various components of IMD 12. Power source 50 may include a small rechargeable or non-rechargeable battery and a power management circuit to produce the operating power. In the case of a rechargeable battery, recharging may be accomplished through proximal inductive interaction between an external charger and an inductive charging coil within IMD 12. In some examples, power requirements may be small enough to allow IMD 12 to utilize patient motion and implement a kinetic energy-scavenging device to trickle charge a rechargeable battery. In other examples, traditional batteries may be used for a limited period of time. As another alternative, an external inductive power supply can transcutaneously power IMD 12 as needed or desired.

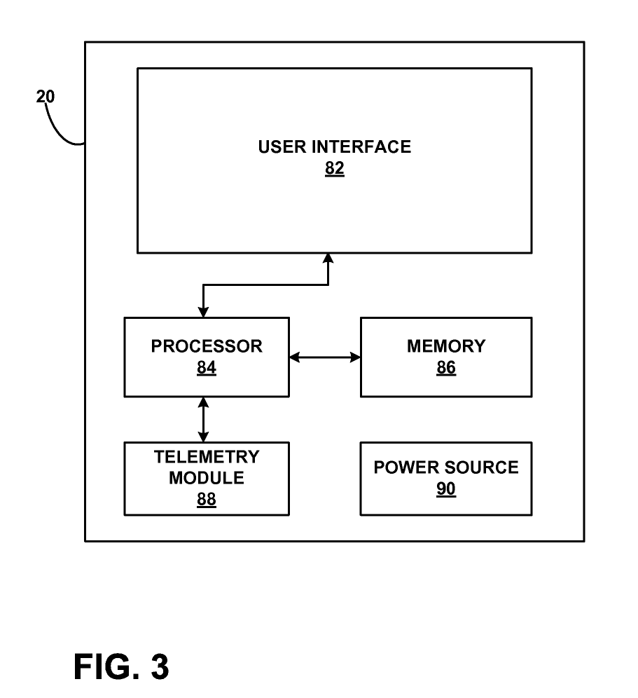

As described, IMD 12 may communicate with one or more external devices at various times during the operation of IMD 12. In the example of FIG. 1, IMD 12 communicates with external programmer 20. FIG. 3 is a functional block diagram illustrating an example of various components of external programmer 20. As shown in FIG. 3, external programmer 20 may include user interface 82, processor 84, memory 86, telemetry module 88, and power source 90. A clinician or patient 16 interacts with user interface 82 to change the parameters of a therapy program, change therapy programs within a group of programs, view therapy information, view historical or establish new therapy programs, or otherwise communicate with IMD 12 or view or edit programming information.

Processor 84 controls user interface 82, retrieves data from memory 86 and stores data within memory 86. Processor 84 also controls the transmission of data through telemetry module 88 to IMD 12. The transmitted data may include, e.g., retrieved sensor data from IMD 12 or instructions assigning particular therapeutic fluids to first reservoir 34 and second reservoir 36. The transmitted data may also include therapy program information specifying various therapeutic fluid delivery parameters. For example, transmitted data may specify, e.g., instructions for actuating first reservoir discharge valve 42 or instructions for controlling fluid delivery pump 32. Memory 86 may store, e.g., operational instructions for processor 84 and data related to therapy for patient 16. Programmer 20 may be a hand-held computing device that includes user interface 82 that can be used to provide input to programmer 20.

User interface 82 may include a display screen or other output media, and user input media. When programmer 20 is configured for use by a clinician, user interface 82 may be used to transmit initial programming information to IMD 12 including hardware information for system 10, e.g. the number of reservoirs 34, 36, the number of fluid delivery pumps 32, the number and type of reservoir discharge valve 42, the position of fluid pathways 48, a baseline orientation of IMD 12 relative to a reference point, and software information related to therapy delivery and operation of IMD 12, e.g., therapy parameters of therapy programs stored within IMD 12 or within programmer 20, the type and amount, e.g., by volume of therapeutic fluid(s) delivered by IMD 12 and any other information the clinician desires to program into IMD 12. Programmer 20 may also be configured to read IMD 12 specific configuration information such as, e.g., the capacity of first reservoir 34 and second reservoir 36, an IMD serial number, calibration information, IMD diagnostic/state information, and the like.

Programmer 20 may also be configured for use by patient 16. When configured as a patient programmer, programmer 20 may have limited functionality in order to prevent patient 16 from altering critical functions or applications that may be detrimental to patient 16, e.g., therapy or dosing parameters. In this manner, programmer 20 may only allow patient 16 to adjust certain therapy parameters or to set an available range for a particular therapy parameter. In some cases, a patient programmer may permit the patient to control IMD 12 to deliver a supplemental, patient activated bolus, if permitted by the applicable therapy program administered by the IMD, e.g., if delivery of a patient bolus would not violate a lockout interval or maximum dosage limit. Programmer 20 may also provide an indication to patient 16 when therapy is being delivered or when IMD 12 needs to be refilled, when the IMD is not operating properly, or when the power source within programmer 20 or IMD 12 need to be replaced or recharged.

Telemetry module 88 allows the transfer of data to and from programmer 20 and IMD 12, as well as other devices, e.g. according to the communication techniques described above with reference to FIG. 2. Power source 90 may be a non-rechargeable battery or rechargeable battery, such as a lithium ion or nickel metal hydride battery. In some examples, programmer 20 may be configured to recharge IMD 12 in addition to programming IMD 12.

FIGS. 4A-4C are cross-sectional views of an example IMD 100. FIG. 4A is a top view of IMD 100. FIG. 4B is a cross-sectional view of IMD 100 taken along section A-A of FIG. 4A. FIG. 4C is an example multilayer structure of an example reservoir of IMD 100. IMD 100 may be implanted in patient 16 in addition to, or in lieu of, IMD 12. IMD 100 may correspond substantially to IMD 12 (FIGS. 1 and 2) and may include additional components illustrated and described with respect to IMD 12. IMD 100 may also communicate with programmer 20 (FIGS. 1 and 3) or another external device communicatively coupled to IMD 100.

Referring to FIG. 4A, IMD 100 includes housing 102 that defines first protrusion 104, second protrusion 106, and third protrusion 108, each of which extend from a center portion 103 of housing 102. IMD 100 also includes first catheter port 110, second catheter port 112, first catheter access port 114, second catheter access port 116, first inlet port 118, and second inlet port 120. First catheter port 110 and second catheter port 112 are configured to connect to catheters for delivering therapeutic fluid to one or more target delivery sites within patient 16. First catheter access port 114 and second catheter access port 116 are in fluid communication with first catheter port 110 and second catheter port 112, respectively. First inlet port 118 is in fluid communication with a first reservoir (not shown). Second inlet port 120 is in fluid communication with a second reservoir (not shown).

In some examples, the components and operation of IMD 100 may correspond to the description of the components and operation of IMD 12 (FIGS. 1 and 2). In some examples, fluid is added and withdrawn from fluid reservoirs of IMD 100 through first inlet port 118 and second inlet port 120 using a fluid delivery needle, e.g., percutaneously inserted into patient 16. In some examples, a user may desire to directly add or remove fluid through first catheter port 110 and/or second catheter port 112, e.g., attached to catheters in fluid communication with patient 16. For example, a user may want to provide a direct fluid injection to patient 16, such as direct injection of therapeutic fluid or a direct injection of a dye for dye study testing.

Alternatively, a user may want to remove fluid from patient 16 for testing and analysis. To accommodate different situations, IMD 100 includes first catheter access port 114 in direct fluid communication with first catheter port 110 and second catheter access port 116 in direct fluid communication with second catheter port 112. Direct fluid communication means that fluid passes through IMD 100 without passing through a reservoir and/or fluid delivery pump of IMD 100. In various examples, first catheter access port 114 and second catheter access port 116 may be configured similar to inlet port 38, 40, discussed above with respect to FIG. 2. In this manner, IMD 100 is configured to provide direct fluid access to patient 16 via first catheter access port 114 and second catheter access port 116.

When accessing IMD 100, it may be useful for the safe and intended operation of IMD 100 if a user, such as a patient or clinician, can readily distinguish between different ports that are connected to different fluid pathways. In some examples, a user may employ an external aid, such as a template or electronic port finder, to identify and distinguish between different ports on IMD 100. In other examples, the user may employ sensors within the IMD 100 to provide confirmation via telemetry as to which access port a needle is inserted into. In yet other examples, the user may rely on the physical geometry of IMD 100 and tactile feel to distinguish between different ports on the fluid delivery device. In the example of FIG. 4A, IMD 100 includes protrusions 104, 106, and 108 that extend from a center portion 103 of housing 102. Protrusions 104, 106, and 108 are asymmetrically arranged to allow a user to distinguish one protrusion from another protrusion based on tactile feel. In addition, different ports (e.g., first catheter access port 114, second catheter access port 116, first inlet port 118, second inlet port 120) are arranged on different protrusions 104, 106, 108, or different positions on the same protrusion 106, thus allowing the user to distinguish the different ports. Hence, an orientation of the housing may be perceptible by a user based on tactile feel of at least three protrusions extending from a center of the housing. In different examples, IMD 100 may include a different number of protrusions, a different number of ports 114, 116, 118, 120, or a different arrangement of ports 114, 116, 118, 120 relative to protrusions 104, 106, 108. The arrangement and location of different protrusions and ports is not critical provided that a user can distinguish an orientation of housing 102 and distinguish different ports from one another. For example, in another example according to the disclosure, IMD 100 may only include two protrusions. First catheter access port 114 and first inlet port 118 may be arranged on one protrusion while second catheter access port 116 and second inlet port 120 may be arranged on another protrusion. In other examples, IMD 100 may include more than three protrusions, such as four, five, or more protrusions.

Alternatively, in some examples, at least one of first catheter access port 114, second catheter access port 116, first inlet port 118 and second inlet port 120 may be located on a part of housing 102 other than protrusions 104, 106, 108. For example, at least one of first catheter access port 114, second catheter access port 116, first inlet port 118 and second inlet port 120 may be located on a center portion 103 of housing 102. In some examples, first catheter access port 114, second catheter access port 116, first inlet port 118 and second inlet port 120 may each be located on a center portion 103 of housing 102, and housing 102 may or may not include one or more protrusions 104, 106, 108 that facilitate distinguishing an orientation of housing 102 and distinguishing different ports 114, 116, 118, 120 from one another.

In some examples, instead or in addition to including one or more protrusions 104, 106, 108, housing 102 may define a shape that permits distinguishing an orientation of housing 102 when implanted in a body of a patient. For example, housing 102 may define an elongated shape (e.g., longer in a first direction than in a second, substantially perpendicular direction), an asymmetrical shape, or the like, which permits distinguishing the orientation of housing 102 when implanted in a body of a patient. Alternatively or additionally, in some examples, first catheter port 110 and second catheter port 112 may be disposed in the same or different ones of protrusions 104, 106, or 108, while first catheter access port 114, second catheter access port 116, first inlet port 118, and/or second inlet port 120 are disposed on housing 102 (e.g., center portion 103 of housing 102).

In some examples, catheter access ports 114, 116 and inlet ports 118, 120 are configured to receive differently sized fluid delivery needles to prevent a user from inadvertently accessing the wrong port. In one example, catheter access ports 114, 116 are configured to receive a fluid delivery needle with a smaller diameter than a fluid delivery needle inlet ports 118, 120 are configured to receive. In various examples, inlet ports 114, 116 are configured to permit a fluid delivery needle larger than or equal to approximately 22 gauge (Outer Diameter (OD) of 0.711 mm) to enter inlet ports 114, 116 while catheter access ports 114, 116 are configured to block the same needle. Catheter access ports 114, 116 may be configured to permit entry of a fluid delivery needle smaller than or equal to approximately 24 gauge (OD 0.559 mm). In some examples, first inlet port 114 may also be configured to receive a different size fluid delivery needle than second inlet port 116. In one example, first inlet port 114 is in fluid communication with a pressurized fluid reservoir, and second inlet port 116 is in fluid communication with a reservoir that is connected to a fluid delivery pump. In this example, first inlet port 114 may be configured to receive a smaller fluid delivery needle than second inlet port 116.

FIG. 4B is a cross-sectional view of IMD 100 taken along the A-A cross-sectional line illustrated in FIG. 4A. IMD 100 in FIG. 4B includes previously described housing 102, first protrusion 104, third protrusion 108, first catheter port 110, and second catheter port 112. Housing 102 defines a first surface 130 and a second surface 132 opposite first surface 130. Housing 102 contains first fluid reservoir 136, first propellant reservoir 138, second reservoir 140, second propellant reservoir 142, fluid delivery pump 144, first reservoir discharge valve 146, and fluid pathways 148, 150, 152, 154. First surface 130 of housing 102 defines a dome-like structure 160 that substantially contains first fluid reservoir 136. Second fluid reservoir 140 may be a bellows reservoir defined by convolution 162. IMD 100 also includes bulkhead 134. Bulkhead 134 houses various components of IMD 100 including, e.g., a memory, processor, telemetry module, power source, and the like. In other examples, dome-like structure 160 may substantially contain second fluid reservoir 140, and first fluid reservoir 136 may be provided elsewhere, such as, for example, adjacent second surface 132. Also, in various examples, first fluid reservoir 136 and second fluid reservoir 140 may be configured as collapsible reservoirs, bellows reservoirs, fixed volume reservoirs, or other types of reservoirs. For example, a collapsible reservoir or a bellows type reservoir could be used to deliver fluid passively or could be coupled to a pump for active delivery. If first fluid reservoir 136 or second fluid reservoir 140 is provided in dome-like structure 160, the respective reservoir may be formed as a collapsible reservoir.

In some examples, the configuration and operation of components illustrated in the example of FIG. 4B correspond to the description of like components in the example of FIG. 2. During operation of IMD 100, a processor controls first reservoir discharge valve 146, e.g., with the aid of instructions stored in a memory of IMD 100. First reservoir discharge valve 146 actuates open, allowing therapeutic fluid to flow from first fluid reservoir 136 through fluid pathway 148, first reservoir discharge valve 146, fluid pathway 150, and first catheter port 110 for delivery to patient 16. In addition to or in lieu of fluid delivery from first fluid reservoir 136, a processor in IMD 100 controls fluid delivery pump 144 to draw from fluid pathway 152. Fluid pathway 152 is in fluid communication with second fluid reservoir 140. Fluid delivery pump 144 pressurizes the therapeutic fluid and discharges the therapeutic fluid through fluid pathway 154 and second catheter port 112 to patient 16.

In some examples, first fluid reservoir 136 and second fluid reservoir 140 may be arranged in numerous locations within IMD 100 including, e.g., adjacent catheter ports 110, 112. In some examples, first fluid reservoir 136 and second fluid reservoir 140 are in a stacked arrangement (e.g., one on top of another in the Y-direction as in the example of FIG. 4B). In other examples, first fluid reservoir 136 and second fluid reservoir 140 are in a coplanar arrangement (e.g., side-by-side in the X-direction shown on FIG. 4B) to minimize the overall thickness of IMD 100.

In some examples, bulkhead 134 may be stacked adjacent second surface 132, and first fluid reservoir 136 and second fluid reservoir 140 may be stacked adjacent each other between bulkhead 134 and first surface 130. This arrangement may facilitate access and manufacturability of electronics in the bulkhead 134 or between bulkhead 134 and surface 132, and/or may facilitate the use of a common propellant chamber or two propellant chambers joined with a pathway and allow a single operation to fill the propellant, possibly making production easier and less costly. Continuing with the example, first fluid reservoir 136 and second fluid reservoir 140 may be in a stacked arrangement or a co-planar arrangement when located between bulkhead 134 and first surface 130. In a co-planar arrangement, reservoirs 136, 140 may be disposed side by side with one another in generally a common plane and have a common or similar height in a direction extending from surface 132 to surface 130. In a stacked arrangement, reservoirs 136, 140 may be disposed one above the other, generally in different planes, i.e., at different levels in a direction extending from surface 132 to surface 130. In different examples, IMD 100 includes more than two reservoirs (e.g., three, four, five, or more reservoirs) to provide additional flexibility for storing different fluids in different reservoirs.

First fluid reservoir 136 is configured to house a therapeutic fluid for passive delivery to patient 16. In the example of FIG. 4B, first fluid reservoir 136 is defined by a collapsible bladder (e.g., a structure that expands and contracts) within a cavity defined by dome-like structure 160. Thus, the volume of first fluid reservoir 136 varies based on the amount of therapeutic fluid in first fluid reservoir 136. In other examples, first fluid reservoir 136 may define a fixed volume that does not vary according to an amount of therapeutic fluid within the reservoir, or may be another type of reservoir, such as a bellows reservoir. Regardless, in the example of FIG. 4B, IMD 100 includes first propellant reservoir 138 adjacent to first fluid reservoir 136. First propellant reservoir 138 configured to house a propellant, e.g., to pressurize therapeutic fluid in first reservoir 136. Propellant is generally a compressible gas that may include, e.g., perfluoropentane, perfluorohexane, or butane. In operation, propellant in first propellant reservoir 138 biases against first fluid reservoir 136 in the direction indicated by arrows 164 to pressurize therapeutic fluid in first fluid reservoir 136, enabling IMD 100 to passively deliver therapeutic fluid from first fluid reservoir 136 to patient 16. The propellant in first propellant reservoir 138 may be configured to apply a substantially constant pressure to first fluid reservoir 136 to passively transfer the first therapeutic fluid to the patient.

Second fluid reservoir 140 is configured to house a therapeutic fluid for active delivery to patient 16. In the example of FIG. 4B, second fluid reservoir 140 is a bellows reservoir defined by convolution 162. In other examples, second fluid reservoir 140 may be a different type of reservoir, e.g., a collapsible bladder or reservoir that defines a fixed volume that does not vary according to an amount of therapeutic fluid within the reservoir. Second propellant reservoir 142 is adjacent to second fluid reservoir 140 and configured to house a propellant. Propellant in second propellant reservoir 142 biases against second fluid reservoir 140 in the direction indicated by arrows 166 in FIG. 4B to create positive pressure in second fluid reservoir 140, e.g., to convey fluid from second fluid reservoir 142 to fluid delivery pump 144. In some examples, the propellant in second propellant reservoir 142 may have the same or a different chemical composition as the propellant in first propellant reservoir 138. In some examples, first propellant reservoir 138 and second propellant reservoir 142 share a common propellant source (not shown), e.g., that pressurizes therapeutic fluids in first fluid reservoir 136 and second fluid reservoir 140 to substantially equal pressures. In some implementations, first propellant reservoir 138 and second propellant reservoir 142 may not be separate, and may comprise a common reservoir (e.g., first propellant reservoir 138 and second propellant reservoir 142 may form a single, unitary reservoir or may include at least one channel or fluidic connection between first propellant reservoir 138 and second propellant reservoir 142). The common reservoir formed by first propellant reservoir 138 and second propellant reservoir 142 in some examples may exert a common, substantially similar pressure on first fluid reservoir 136 and second fluid reservoir 140. The substantially similar pressure exerted on first fluid reservoir 136 and second fluid reservoir 140 by a common reservoir may be a substantially constant pressure.

Alternatively, in other examples, IMD 100 includes a second propellant reservoir 142 that maintains neutral pressure and fluid is housed in second fluid reservoir 140 at neutral pressure, e.g., atmospheric pressure. In another example, fluid is housed in second fluid reservoir 140 at a pressure less than atmospheric pressure and hence the propellant in second propellant reservoir 142 may be at a pressure lower than atmospheric pressure and/or lower than the pressure of first propellant reservoir 138. In some examples, when fluid is housed in second fluid reservoir 140 at a pressure lower than atmospheric pressure, no propellant may be used in second propellant reservoir.

While IMD 100 includes first propellant reservoir 138 and second propellant reservoir 142, in different examples, one or both of propellant reservoirs 138, 142 is replaced with a different biasing means including, e.g., a spring, hydraulic piston, or similar biasing means. Further, while FIG. 4B illustrates first fluid reservoir 136 as a collapsible bladder and second fluid reservoir 140 as a bellows reservoir, first fluid reservoir 136 and second fluid reservoir 140 may be any components or set of components configured to house therapeutic fluids for delivery to patient 16.

In some examples, first fluid reservoir 136, second fluid reservoir 140, first propellant reservoir 138, and second propellant reservoir 142 are constructed of materials that resist corrosion and degradation from, e.g., therapeutic fluids, propellant, and bodily fluids. Example materials include biocompatible metals, e.g., stainless steel, titanium, nickel-titanium alloy such as nitinol or the like, and biocompatible polymers, e.g., polyether ether ketone (PEEK), silicone or silane based polymers, various elastomers, e.g., polyethylene, polypropylene, polystyrene, or the like. In one example, first fluid reservoir 136 and second fluid reservoir 140 are constructed of titanium. In another example, first fluid reservoir 136 and/or second fluid reservoir 140 are constructed of multiple materials.

Although the example IMD 100 shown in FIGS. 4A and 4B includes a single housing 102, in other examples, IMD 100 may include at least two housings. For example, first fluid reservoir 136 (along with first propellant reservoir 138) may be contained in a first housing and second fluid reservoir 140 (along with second propellant reservoir 142) may be contained in a second housing. In some examples, the first housing and the second housing may be at least partially encapsulated by a common member, which couples the first housing and the second housing to form IMD 100. In some examples, the member may be constructed of a biologically inert polymer, such as a silicone or a polyurethane. The member may be substantially rigid or may be flexible. In some examples, at least a portion of the first housing and/or the second housing may be exposed to the external environment (e.g., may not be encapsulated by the member), while in other examples, the member may substantially fully encapsulate both the first housing and the second housing.

FIG. 4C is an example multilayer structure 178 used to construct an example collapsible bladder for first fluid reservoir 136. Multilayer structure 178 includes a first layer 180, a second layer 182, and a third layer 184. Second layer 182 is interposed between first layer 180 and third layer 184. In some examples, second layer 182 is constructed of a pliable material, e.g., to allow first fluid reservoir 136 to expand and contract as fluid is added and withdrawn from first fluid reservoir 136. As such, second layer 182 may be a flexible membrane. In one example, second layer 182 is constructed of an elastomer. Suitable elastomers may include, but are not limited to, ethylene propylene rubber, silicon rubber, fluoro and perfluoro elastomers, and the like. In some examples, first layer 180 is disposed adjacent a propellant housed in first propellant reservoir 138, while third layer 184 is disposed adjacent a therapeutic fluid housed in first fluid reservoir 136. In any event, first layer 180 and/or third layer 184 may be constructed of one or more materials substantially impermeable to, and unreactive with, propellant and/or therapeutic fluid. In one example, first layer 180 and/or third layer 184 comprise a metalized film formed over second layer 182. In another example, first layer 180 and/or third layer 184 are formed by coating a protective film over second layer 182, e.g., resistant to therapeutic fluid and/or propellant. Multilayer structure 178 may be used to form a wall of an example collapsible bladder.

With further reference to FIG. 4B, IMD 100 includes housing 102. In some implementations, housing 102 is constructed of a biocompatible material that, e.g., resists degradation when exposed to bodily fluids and that cannot be punctured by an inadvertent needle prick during a therapeutic fluid refilling operation. Housing 102 defines first surface 130 and second surface 132 opposite first surface 130. Housing 102 is configured to house various components of IMD 100 and may be any suitable shape. In one example, first surface 130 of housing 102 defines dome-like structure 160 that may include, e.g., a convex shape. IMD 100 can be implanted in patient 16 with the skin of patient 16 draping over dome-like structure 160. Dome-like structure 160 does not present sharp housing lines normally associated with implanted medical devices. As a result, dome-like structure 160 provides patient 16 with an aesthetically pleasing implanted medical device. That is, IMD 100 provides a smooth appearance under the skin of patient 16 following implantation, as opposed to an IMD that provides sharp lines following implantation. Further, dome-like structure 160 may avoid skin irritation and tissue erosion on patient 16. In some examples, first surface 130 defines a convex shape and second surface 132 defines a concave shape, e.g., to provide a conformable, aesthetically pleasing shape for implanting IMD 100 in patient 16.

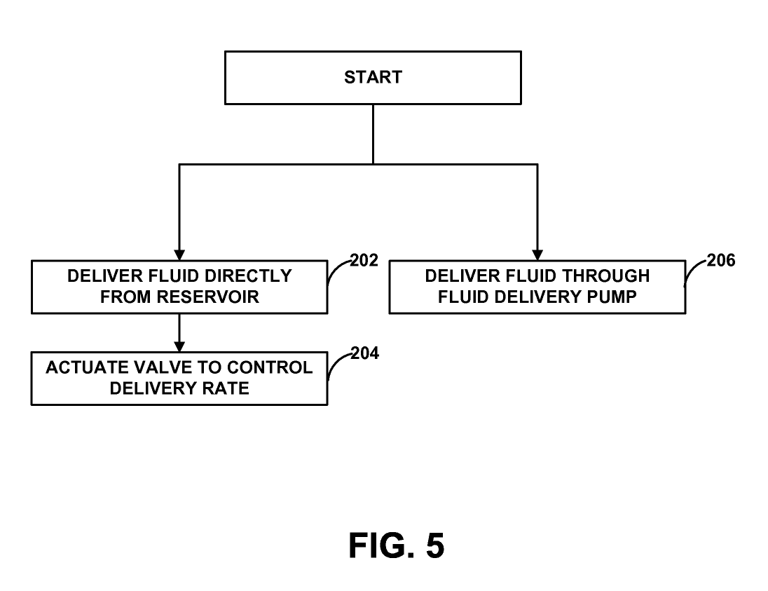

Different IMD configurations and fluid delivery configurations have been described in relation to FIGS. 1-4. In different examples, the various IMD configurations can be used to deliver therapeutic fluid to patient 16. FIG. 5 is a flow chart illustrating an example method of delivering therapeutic fluid with an example implantable fluid delivery device. The method of FIG. 5 includes passively delivering therapeutic fluid directly from a reservoir (202), and actively delivering therapeutic fluid through a fluid delivery pump (206). In some examples, the method of FIG. 5 also includes actuating a valve to control the delivery rate of the therapeutic fluid delivered directly from the reservoir (204). For ease of description, the functions of the method of FIG. 5 for delivering fluid from an IMD are described as executed by IMD 12 (FIG. 2). In other examples, however, the method of FIG. 5 may be executed by IMD 100 or IMDs with different configurations, as described herein.

The method of FIG. 5 includes passively delivering therapeutic fluid directly from a reservoir (202). In one example, therapeutic fluid is delivered from first reservoir 34 to patient 16 immediately upon adding therapeutic fluid to first reservoir 34. In another example, programmer 20, or another device communicatively coupled to IMD 12, transmits instructions through telemetry module 88 and telemetry module 30 to actuate first reservoir discharge valve 42 open. Processor 26 in IMD 12 receives the instructions and transmits a command to actuate first reservoir discharge valve 42. In some examples, processor 26 of IMD 12 transmits a confirmation message back to programmer 20 indicating that first reservoir discharge valve 20 was actuated open, e.g., for storage in memory 86 or to provide an indication via user interface 82 informing the user that fluid is being delivered from first reservoir 34. In further examples, processor 26 executes instructions stored in memory 28 to actuate first reservoir discharge valve 42, e.g., according to a therapy program that provides set dosing rates or a set dosing schedule for delivering therapeutic fluid from first reservoir 34. Regardless of how the process is initiated, in various examples, IMD 12 is configured to deliver therapeutic fluid directly from first reservoir 34. In some examples, the therapeutic fluid may be delivered at a substantially constant rate, e.g., based on a substantially constant pressure applied to first reservoir 34.

In some examples, IMD 12 is configured to control the passive fluid delivery rate from first reservoir 34. For example, first reservoir discharge valve 42 may actuate to a plurality of different settings, e.g., to change the fluid flow rate passing through fluid pathways 48C and 48D from first reservoir 34. In some examples, first reservoir discharge valve 42 is configured to actuate to any position between fully open and fully closed. In other examples, first reservoir discharge valve 42 is configured to actuate to discrete number of settings. In either configuration, the method of FIG. 5 includes, in various examples, actuating first reservoir discharge valve to control the delivery rate of the therapeutic fluid delivered directly from first reservoir 34 (204). In one example, programmer 20, or another device communicatively coupled to IMD 12, transmits instructions to processor 26 in IMD 12 to actuate first reservoir discharge valve 42 to a specific position. In some examples, instructions specify a target valve position, e.g., “seventy-five percent open.” In other examples, instructions specify a specific fluid dosing rate, e.g., “eight microliters per hour,” which must be analyzed, e.g., compared to a look-up table stored in memory 28, to determine a valve position based on the specified instructions. In additional examples, processor 26 actuates first reservoir discharge valve 42 based on instructions stored in memory 28. In one example, the instructions define a therapy program, e.g., that provides a schedule of different dosing rates for different times of the day or a schedule of different valve settings for different times of the day.

In conjunction with or in lieu of delivering the therapeutic fluid directly from a reservoir (202), the method of FIG. 5 includes actively delivering therapeutic fluid through a fluid delivery pump (206). In the configuration of IMD 12, therapeutic fluid from second reservoir 36 may be actively delivered through fluid delivery pump 32 to patient 16. In one example, programmer 20, or another device communicatively coupled to IMD 12, sends instructions to processor 26 to activate fluid delivery pump 32. Fluid delivery pump 32 activates in response to instructions from processor 26, drawing fluid from second reservoir 36. Mechanical energy is imparted into the fluid from second reservoir 36 as the fluid passes through fluid delivery pump 32, resulting in fluid transfer from second reservoir 36 to patient 16. In another example, processor 26 executes instructions stored in memory 28 to activate fluid delivery pump 32, e.g., according to a therapy program the provides set dosing rates or a set dosing schedule for delivering therapeutic fluid from second reservoir 36. In various examples, processor 26 may control fluid delivery pump 32, e.g., to increase or decrease the rate fluid delivery rate through fluid delivery pump 32.

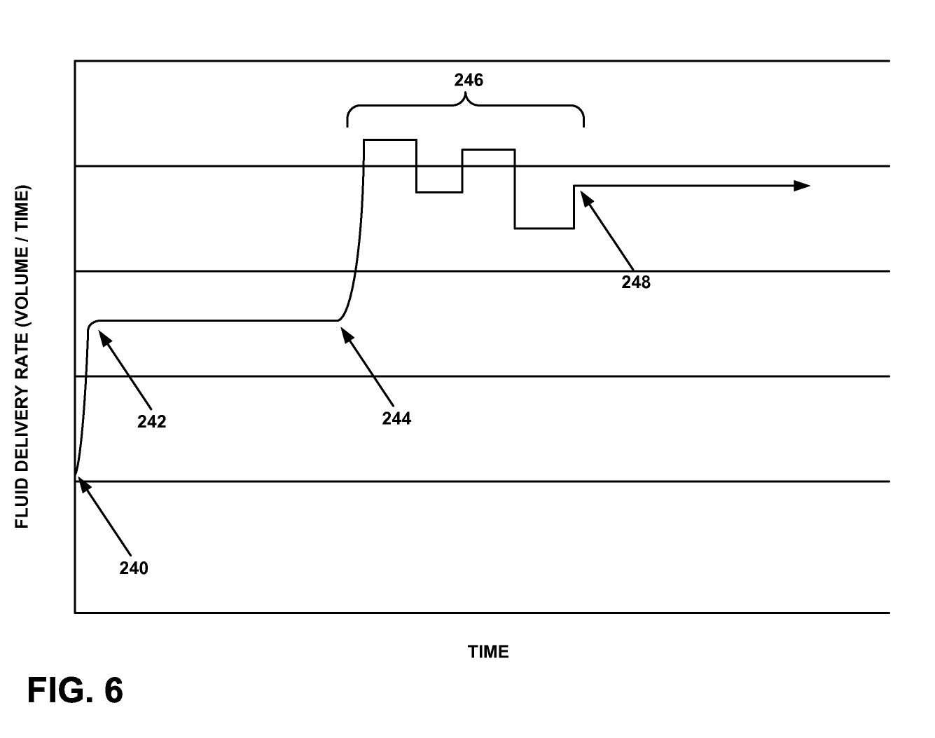

The foregoing fluid delivery methods and fluid delivery device configurations can be used to provide a variety of fluid therapies. FIG. 6 is an example graph of example fluid delivery rates provided by IMD 12 versus time. FIG. 6 illustrates cumulative fluid delivery rates, i.e., the total rate of fluid passively delivered from first reservoir 34 and actively delivered from second reservoir 36, which may be the same fluid in each reservoir 34, 36 or different fluids in each reservoir 34, 36. According to the example of FIG. 6, fluid delivery starts at an initial rate at time 240 and ramps up to a substantially constant basal rate at time 242. In some examples, where IMD 12 includes first reservoir discharge valve (e.g., FIG. 2), the basal delivery rate established at time 242 may be determined by a position of first reservoir discharge valve 42. Accordingly, the fluid delivery rate may change relatively rapidly as first reservoir discharge valve 42 is actuated between the first state (at time 240) and the second state (at time 242). In alternative examples, fluid is actively delivered from second reservoir 36 and fluid delivery pump 32 in addition to, or instead of, from first reservoir 34.

In the example of FIG. 6, fluid is delivered at a continuous rate between time 242 and time 244. At time 244, the fluid delivery rate escalates and enters a regime of variable fluid delivery rates over time 246. In one example, during time 246, fluid is passively delivered from first reservoir 34 at the rate indicated between time 242 and time 244 with additional fluid actively provided from second reservoir 36 through fluid delivery pump 32. Hence, in some examples, fluid may be passively delivered from first reservoir 34 to provide a baseline rate of fluid delivery, and fluid can be actively delivered from second reservoir 36 to selectively add to the baseline rate of fluid delivery, providing a cumulative rate of fluid delivery that may be varied by varying the rate of actively delivered fluid. In another example, first reservoir discharge valve 42 closes at time 244 and fluid delivery pump 32 provides all fluid delivery during time 246. In either example, fluid delivery pump 32 provides variable, active fluid delivery rates during time 246 which may, e.g., be dictated by therapy delivery programs stored in memory 28.

At time 248, fluid delivery returns to a constant basal rate which, in the example of FIG. 6, is higher than the constant fluid delivery rate established between time 242 and time 244. In some examples, fluid delivery pump 32 shuts down at time 248 and fluid is delivered solely from first reservoir 34. In one example, first reservoir discharge valve 42 actuates at time 248 to increase the fluid delivery rate from first reservoir 34. In this manner, first reservoir 34 is capable of delivering the increased fluid delivery rate at time 248 relative to the rate delivered between time 242 and time 244. In another example, first reservoir 32 delivers fluid at time 248 at the rate established between time 242 and time 244. Second reservoir 36 and fluid delivery pump 32 provide the additional fluid delivered at time 248. According to another example, first reservoir discharge valve 42 actuates closed at time 248 and fluid is delivered solely from second reservoir 36 via fluid delivery pump 32 at time 248. Regardless, in the example of FIG. 6, first reservoir 34 and second reservoir 36 may house the same therapeutic fluid or different therapeutic fluids, e.g., to treat different medical conditions or to more effectively treat a single medical condition.

While in the preceding examples a target therapy delivery site(s) was described as being proximate to the spinal cord of a patient, other applications of therapy systems in accordance with this disclosure include alternative delivery sites. In some examples, the target delivery site may be proximate to different types of tissues including, e.g., nerves, e.g. sacral, pudendal or perineal nerves, organs, muscles or muscle groups. In one example, a catheter may be positioned to deliver a therapeutic fluid to a deep brain site or within the heart or blood vessels. Delivery of a therapeutic fluid within the brain may help manage a number of disorders or diseases including, e.g., chronic pain, depression or other mood disorders, dementia, obsessive-compulsive disorder, migraines, obesity, and movement disorders, such as Parkinson's disease, spasticity, and epilepsy. A catheter may also be positioned to deliver insulin to a patient with diabetes. In other examples, the system may deliver a therapeutic fluid to various sites within a patient to facilitate other therapies and to manage other conditions including peripheral neuropathy or post-operative pain mitigation, ilioinguinal nerve therapy, intercostal nerve therapy, gastric drug induced stimulation for the treatment of gastric motility disorders and/or obesity, and muscle stimulation, or for mitigation of peripheral and localized pain e.g., leg pain or back pain. In still other examples, the system may deliver different therapeutic fluids to different target therapy sites to manage multiple different medical conditions. For example, the system may deliver a cancer treatment therapeutic fluid (e.g., a chemotherapy agent) to a tumor site while delivering a different therapeutic fluid (e.g., an analgesic) to an intrathecal space for pain management.

Various aspects of the techniques described in this disclosure may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in a non-transitory computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

1. An implantable therapeutic fluid delivery device comprising:
a first reservoir, configured to house a first therapeutic fluid, the first reservoir configured to passively transfer the first therapeutic fluid to a patient;
a second reservoir configured to house a second therapeutic fluid; and
a fluid delivery pump configured to actively transfer the second therapeutic fluid from the second reservoir to the patient.

2. The implantable therapeutic fluid delivery device of claim 1, further comprising a first catheter port configured to connect to a catheter, and a second catheter port configured to connect to the catheter, wherein the first reservoir is configured to passively transfer the first therapeutic fluid to the first catheter port, and the fluid delivery pump is configured to actively transfer the second therapeutic fluid to the second catheter port.

3. The implantable therapeutic fluid delivery device of claim 2, wherein the catheter comprises a first catheter and a second catheter separate from the first catheter, the first catheter port configured to connect to the first catheter, and the second catheter port configured to connect to the second catheter.

4. The implantable therapeutic fluid delivery device of claim 1, further comprising a propellant reservoir, wherein a propellant in the propellant reservoir is configured to apply a substantially constant pressure to the first reservoir to passively transfer the first therapeutic fluid to the patient.

5. The implantable therapeutic fluid delivery device of claim 4, wherein the propellant reservoir is configured to apply a substantially constant pressure to the first reservoir and the second reservoir.

6. The implantable therapeutic fluid delivery device of claim 4, wherein the first reservoir defines a collapsible bladder.

7. The implantable therapeutic fluid delivery device of claim 6, wherein the collapsible bladder comprises a first layer, a second layer, and a third layer, the second layer interposed between the first layer and the third layer, and wherein the first layer and the third layer comprise a metal, and the second layer comprises a flexible membrane.

8. The implantable therapeutic fluid delivery device of claim 6, wherein the second reservoir defines a bellows reservoir.

9. The implantable therapeutic fluid delivery device of claim 6, wherein the housing defines a first surface and a second surface opposite the first surface, wherein the first surface defines a dome-like structure, and wherein the first reservoir is substantially contained in a cavity defined by the dome-like structure.

10. The implantable therapeutic fluid delivery device of claim 1, further comprising at least one of a valve or a restrictor interposed between the first reservoir and the patient.

11. The implantable therapeutic fluid deliver device of claim 10, wherein the valve is configured to actuate to a plurality of different settings, and the first reservoir is configured to transfer the first therapeutic fluid to the patient at one of a plurality of different substantially constant rates, the one of the plurality of different substantially constant rates being based on the setting of the valve.

12. The implantable therapeutic fluid delivery device of claim 1, wherein the housing defines at least three protrusions extending from a center of the housing.

13. The implantable therapeutic fluid delivery device of claim 12, further comprising:
a first catheter access port configured for fluid communication with a first catheter, wherein the first reservoir is configured to passively transfer the first therapeutic fluid to the first catheter;
a second catheter access port configured for fluid communication with a second catheter, wherein the fluid delivery pump is configured to actively transfer the second therapeutic fluid from the second reservoir to the second catheter;
a first inlet port configured to receive a fluid delivery needle, wherein the first inlet port is configured for fluid communication with the first reservoir; and
a second inlet port configured to receive the fluid delivery needle, wherein the second inlet port is configured for fluid communication with the second reservoir,
wherein the first catheter access port, the second catheter access port, the first inlet port, and the second inlet port are located on the at least three protrusions.

14. The implantable medical device of claim 1, further comprising a housing, wherein the housing contains the first reservoir, the second reservoir, and the fluid delivery pump.

15. A method comprising:
passively delivering a first therapeutic fluid to a patient from a first reservoir configured to house the first therapeutic fluid; and
actively delivering a second therapeutic fluid to the patient from a second reservoir configured to house the second therapeutic fluid,
wherein an implantable medical device includes the first and second reservoirs.

16. The method of claim 15, wherein passively delivering the first therapeutic fluid comprises providing a pressure on the first therapeutic fluid to provide force to transfer the first therapeutic fluid to the patient, and actively delivering the second therapeutic fluid comprises applying a fluid delivery pump to provide force to transfer the second therapeutic fluid to the patient.

17. The method of claim 15, wherein delivering the first therapeutic fluid to the patient comprises delivering the first therapeutic fluid from the first reservoir to a catheter via a first catheter access port configured to connect to the catheter via a first catheter port, and delivering the second therapeutic fluid to the patient from the second reservoir comprises delivering the second therapeutic fluid to the catheter via a second catheter access port configured to connect to the catheter via a second catheter port.

18. The method of claim 17, wherein the catheter comprises a first catheter and a second catheter separate from the first catheter, wherein the first catheter access port is configured to connect to the first catheter via the first catheter port, and wherein the second catheter access port is configured to connect to the second catheter via the second catheter port.

19. The method of claim 15, wherein a propellant in a common propellant chamber applies a substantially constant pressure to both the first reservoir and the second reservoir.

20. The method of claim 15, wherein a propellant in a propellant reservoir applies a substantially constant pressure to the first reservoir to pressurize the first therapeutic fluid.

21. The method of claim 20, wherein the first reservoir defines a collapsible bladder.

22. The method of claim 21, wherein the collapsible bladder comprises a first layer, a second layer, and a third layer, the second layer interposed between the first layer and the third layer, and wherein the first layer and the third layer comprise a metal, and the second layer comprises a flexible membrane.

23. The method of claim 21, wherein the second reservoir defines a bellows reservoir.

24. The method of claim 21, wherein the implantable medical device comprises a housing defining a first surface and a second surface opposite the first surface, the first surface defining a dome-like structure, wherein the first reservoir is substantially contained in a cavity defined by the dome-like structure.

25. The method of claim 15, wherein the implantable medical device comprises a housing including at least one of a valve or a restrictor interposed between the first reservoir and the patient.

26. The method of claim 25, wherein the housing comprises the valve, wherein the method further comprises actuating the valve to one of a plurality of different settings, and wherein delivering the first therapeutic fluid to the patient comprises delivering the first therapeutic fluid to the patient at one of a plurality of different substantially constant rates based on the setting of the valve.

27. The method of claim 15, wherein an orientation of the housing is perceptible based on tactile feel of at least three protrusions extending from a center of the housing.

28. The method of claim 27, wherein:
a first catheter access port, a second catheter access port, a first inlet port, and a second inlet port are located on the at least three protrusions,
the first catheter access port is configured for fluid communication with a first catheter, wherein delivering the first therapeutic fluid comprises delivering the first therapeutic fluid from the first reservoir to the first catheter;
the second catheter access port is configured for fluid communication with a second catheter, wherein delivering the second therapeutic fluid to the patient from the second reservoir comprises delivering the second therapeutic fluid from the second reservoir to the second catheter;
the first inlet port is configured to receive a fluid delivery needle, wherein the first inlet port is configured for fluid communication with the first reservoir; and
the second inlet port is configured to receive the fluid delivery needle, wherein the second inlet port is configured for fluid communication with the second reservoir.

29. The method of claim 15, wherein the implantable medical device includes a fluid delivery pump to provide force to transfer the second therapeutic fluid to the patient, and a housing that contains the first reservoir, the second reservoir, and the fluid delivery pump.

30. An implantable therapeutic fluid delivery device comprising:
means for housing a first therapeutic fluid;
means for housing a second therapeutic fluid;
means for passively delivering the first therapeutic fluid to a patient; and
means for actively delivering the second therapeutic fluid to the patient.

31. The implantable therapeutic fluid delivery device of claim 30, wherein the means for passively delivering the first therapeutic fluid to the patient comprises means for passively delivering the first therapeutic fluid to the patient at a substantially constant rate.

32. The implantable therapeutic fluid delivery device of claim 30, further comprising a first catheter port configured to connect to a catheter and a second catheter port configured to connect to the catheter, wherein the means for housing the first therapeutic fluid is configured for fluid communication with the first catheter port, and wherein the means for housing the second therapeutic fluid is configured for fluid communication with the second catheter port.

33. The implantable therapeutic fluid delivery device of claim 30, wherein the means for passively delivering the first therapeutic fluid to the patient comprise a propellant.

34. The implantable therapeutic fluid delivery device of claim 30, wherein the means for actively delivering the second therapeutic fluid to the patient comprise a fluid delivery pump.

35. The implantable therapeutic fluid delivery device of claim 30, wherein the first therapeutic fluid and the second therapeutic fluid comprise the same therapeutic fluid.
